SEQUENCE LISTING (2 sequences)

SEQ ID NO: 1 - DNA, 27 bp, unidentified
  source 1..27: mol_type = other DNA
acctgggggagtattgcggaggaaggt

SEQ ID NO: 2 - DNA, 72 bp, unidentified
  source 1..72: mol_type = other DNA
atccgtcacacctgctctactggccggctcagcatgactaagaaggaagttatgtggtgttggctcccgtat

(19) United States
(12) Patent Application Publication
(10) Pub. No.: US 2024/0264109 A1
(43) Pub. Date: Aug. 8, 2024

(54) PREPARATION METHOD AND USE OF BiOX/N-DOPED BIOCHAR NANOCOMPOSITE

(71) Applicant: JIANGSU UNIVERSITY, Zhenjiang (CN)

(72) Inventors: Yuting YAN; Hanping MAO; Lizhi LI

(73) Assignee: JIANGSU UNIVERSITY, Zhenjiang (CN)

(21) Appl. No.: 18/249,384

(22) PCT Filed: Jul. 11, 2022
(86) PCT No.: PCT/CN2022/104800

§ 371 Date: Apr. 18, 2023

(30) Foreign Application Priority Data
Jun. 22, 2022 (CN) 202210709707.4

(51) Int. Cl. G01N 27/30; G01N 27/327

(52) U.S. Cl. CPC G01N 27/308; B01J 19/10; B01J 19/124; B82Y 15/00; G01N 27/305; G01N 27/3278; G01N 33/5438; G01N 2021/7753; G01N 2333/245

(57) ABSTRACT

A preparation method and use of a BiOX/N-doped biochar nanocomposite, where X is I or Br is provided. The preparation method includes the following steps: step 1: preparation of an N-doped biochar; step 2: preparation of an acidified N-doped biochar; and step 3: preparation of the BiOX/N-doped biochar nanocomposite. In the present disclosure, a discarded crayfish shell, crab shell, or tofu residue is used as a raw material to prepare the BiOX/N-doped biochar nanocomposite, to realize the transformation of a renewable biological resource from waste into treasure. A photoelectric sensor is constructed based on the BiOX/N-doped biochar nanocomposite that can realize the detection of adenosine triphosphate (ATP) or Escherichia coli (E. coli).

### CROSS REFERENCE TO RELATED APPLICATIONS

This application is the national stage entry of International Application No. PCT/CN2022/104800, filed on Jul. 11, 2022 which is based upon and claims priority to Chinese Patent Application No. 202210709707.4 filed on Jun. 22, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBNJIP108ST26_Sequence_Listing-20230403.xml, created on 04/03/2023, and is 3,014 bytes in size.

### TECHNICAL FIELD

The present disclosure belongs to the field of biochar materials and use thereof, and in particular relates to a BiOX/N-doped biochar nanocomposite, and a preparation method and use thereof.

### BACKGROUND

China is a major power for breeding and eating crayfish and crabs, and tens of thousands of tons of crayfish and crab shells are produced annually in China. These crayfish and crab shells are often treated as litters, which not only causes a great waste, but also brings great harm to the ecological environment. In fact, crayfish and crab shells include a large number of useful chemical substances, such as chitin, proteins, calcium carbonate, and a small number of lipids, but most of the crayfish and crab shells are only used for the extraction of chitin at present. It is necessary to seek for a new recycling way to reduce the waste of crayfish and crab shells.

As a by-product of tofu processing, tofu residue includes rich nutrients, has a crude protein content as high as 25% to 30%, and is one of the cheap feeds for pigs. However, the current scientific use of tofu residue is very limited. China is a large soybean planter, with an annual tofu residue output of about more than 3 million tons. If the tofu residue can be fully utilized, the transformation from waste into treasure can be achieved and the environmental burden can be reduced.

Biochar has a large specific surface area (SSA), well-developed pore structures, and abundant surface functional groups, and exhibits a prominent adsorption capacity for metal ions in water. In addition, biochar can be prepared with easily-available raw materials through a simple process. Therefore, biochar is expected to be used as a cheap adsorbent in actual wastewater treatment. Most of the current studies in this field inside and outside China focus on the preparation and adsorption of biochar, but the application of biochar in other fields is rarely reported.

In recent years, it has been proved that bismuth oxyiodide (BiOI) or bismuth oxybromide (BiOBr) has excellent optical properties due to its prominent energy band structure and unique layered tetragonal structure. However, so far, the research on BiOI or BiOBr has generally focused on the photocatalytic performance, and has rarely involved other application fields.

### SUMMARY

An objective of the present disclosure is to provide a preparation method of a BiOX/N-doped biochar nanocomposite, where a discarded crayfish shell, crab shell, or tofu residue is used as a raw material to prepare the BiOX/N-doped biochar nanocomposite, to realize the transformation of a renewable biological resource from waste into treasure. A use of a BiOX/N-doped biochar nanocomposite prepared by the method in the detection of adenosine triphosphate (ATP) or Escherichia coli (E. coli) by a photoelectrochemical technology is investigated. The BiOX/N-doped biochar nanocomposite prepared by the microwave method can be used as a photoelectric active material to construct a photoelectrochemical sensor, and the photoelectrochemical sensor can be used in the fields of plant nutrient detection and food safety, which broadens the application fields of biochar and BiOX.

The present disclosure is implemented by the following technical solutions.

A preparation method of a BiOX/N-doped biochar nanocomposite is provided, including the following steps:
step 1: preparation of an N-doped biochar placing a cleaned crayfish shell, crab shell, or tofu residue in an aluminum oxide crucible, adding a sufficient amount of a strong alkali, and conducting calcination in a tube furnace with an inert atmosphere; and cooling a resulting system, washing the system until neutral, and collecting and drying a resulting solid to obtain the N-doped biochar;
step 2: preparation of an acidified N-doped biochar dispersing the N-doped biochar obtained in the step 1 in a mixed solution of HCl and HNO₃ to obtain a mixed solution A; subjecting the mixed solution A to an ultrasonic treatment in an ultrasonic cleaner, and filtering; and washing a filter residue, and drying the filter residue in an oven to obtain the acidified N-doped biochar, which is denoted as NBC; and
step 3: preparation of the BiOX/N-doped biochar nanocomposite adding the acidified N-doped biochar obtained in the step 2 and Bi(NO₃)₃·5H₂O to acetic acid, and subjecting a resulting mixture to an ultrasonic treatment to obtain a suspension A; under vigorous stirring, adding a KX aqueous solution dropwise to the suspension A to obtain a mixed solution; continuously stirring the mixed solution, transferring the mixed solution to a CEM microwave reactor, setting a microwave power, and conducting a reaction at a constant temperature; after the reaction is completed, collecting a resulting solid through centrifugation, and washing the solid; and dispersing the solid in absolute ethanol, drying, and subjecting a dried product to calcination in a tube furnace with a N₂ atmosphere to obtain a BiOX/N-doped biochar composite, which is denoted as a BiOX/NBC nanocomposite, where X is I or Br.

In the step 1, the strong alkali is NaOH or KOH; the inert atmosphere is Ar; the calcination is conducted as follows: raising a temperature at 5° C./min from room temperature to 700° C., and holding the temperature for 2 h; and the drying is conducted at 80° C. for 24 h.

In the step 2, in the mixed solution of HCl and HNO₃, a volume ratio of the HCl to the HNO₃ is 3:1; and the ultrasonic treatment is conducted for 6 h.

In the step 3, in the suspension A, the acidified N-doped biochar, the Bi(NO₃)₃·5H₂O, and the acetic acid are used in a ratio of (1-20) mg:(0.01-0.05) mol: 40 mL; and the continuous stirring is conducted for 30 min.

In the step 3, a concentration of KX in the KX aqueous solution is 0.5 mol/L; and a volume ratio of the suspension A to the KX aqueous solution is 2:1.

In the step 3, the reaction is conducted at the constant temperature of 150° C. to 180° C. and the microwave power of 200 W for 1 h; and the calcination in the tube furnace is conducted at 300° ° C. for 2 h.

A use of the BiOX/N-doped biochar nanocomposite prepared by the preparation method of the present disclosure in preparation of a photoelectrochemical sensor for detecting ATP or E. coli is provided.

### A Use of the BiOX/N-Doped Biochar Nanocomposite in the Preparation of a Photoelectrochemical Sensor for Detecting ATP is Provided, Including the Following Steps:

(A1) dispersing the BiOX/N-doped biochar nanocomposite in N,N-dimethylformamide (DMF) to obtain a suspension;
(A2) adding 10 μL to 50 μL of the suspension obtained in the step (A1) to an indium tin oxide (ITO) electrode for modification, drying the ITO electrode at room temperature to obtain a modified electrode, which is denoted as BiOX/NBC/ITO; and drip-coating 10 μL to 50 μL of an ATP aptamer solution on the modified electrode to obtain an aptamer/BiOX/NBC/ITO electrode; and
(A3) drip-coating 10 μL to 50 μL of an ATP solution at different concentrations on the aptamer/BiOX/NBC/ITO electrode to obtain an ATP/aptamer/BiOX/NBC/ITO electrode; and assembling an workstation electrochemical three-electrode system with the ATP/aptamer/BiOX/NBC/ITO electrode as a working electrode, a saturated calomel electrode (SCE) as a reference electrode, and a platinum wire as a counter electrode, and under irradiation of a xenon light source, using the photoelectrochemical sensor constructed based on the BiOI/N-doped biochar nanocomposite to detect ATP.

In the step (A1), a concentration of the BiOX/N-doped biochar nanocomposite in the suspension is 5 mg/mL.

In the step (A2), an ATP aptamer has a sequence of 5′-ACCTGGGGGAGTATTGCGGAGGAAGGT-3′ (SEQ ID NO: 1).

In the step (A3), the ATP solution has a concentration of 1×10⁻¹² mol/L to 1×10⁻⁵ mol/L; and the xenon light source has an intensity of 25% to 100%.

A Use of the BiOX/N-Doped Biochar Nanocomposite in the Preparation of a Photoelectrochemical Sensor for Detecting E. coli is Provided, Including the Following Steps:
(B1) preparation of a BiOX/NBC nanocomposite dispersion dispersing the prepared BiOX/NBC nanocomposite in DMF to obtain the dispersion;
(B2) surface pretreatment of an ITO electrode
boiling a 1×0.5 cm² ITO electrode in a 1 mol/L sodium hydroxide solution for 15 min to 20 min, subjecting the ITO electrode to ultrasonic cleaning with acetone, double-distilled water, and ethanol successively, and blow-drying the ITO electrode with nitrogen for later use;
(B3) construction of a photoelectrochemical biological interface
drip-coating 10 μL to 30 μL of the BiOX/NBC nanocomposite dispersion prepared in the step (B1) on a surface of the ITO electrode obtained in the step (B2), and oven-drying to obtain an electrode denoted as BiOX/NBC/ITO; drip-coating 5 μL to 10 μL of glutaraldehyde (GA) on a surface of the BiOX/NBC/ITO, and adding 6 μL to 10 μL of a 3 μmol/L to 5 μmol/L E. coli O157: H7 aptamer solution to the surface of the electrode for modification to obtain an E. coli O157: H7 aptamer/BiOX/NBC/ITO electrode; refrigerating the electrode overnight at 4° C., rinsing the electrode with PBS, and drying; and drip-coating 5 μL to 10 μL of bovine serum albumin (BSA) (1 mmol/L) on the surface of the electrode, allowing the electrode to stand at room temperature for 1 h to block non-specific adsorption sites on the modified electrode, and finally rinsing the modified electrode with ultrapure water (UPW) to remove the unbound aptamer; and
(B4) correspondence between an E. coli 0157: H7 concentration and a pulsed eddy current (PEC) signal
assembling an electrochemical workstation three-electrode system with the E. coli 0157: H7 aptamer/BiOX/NBC/ITO electrode obtained in the step (B3) placed in PBS (pH=7 to 8, concentration: 0.1 mol/L) as a working electrode at a bias voltage of 0.0 V, a platinum wire electrode as a counter electrode, and an SCE as a reference electrode, and under irradiation of a xenon light source, acquiring a photoelectrochemical signal by an i-t curve method; and immersing the E. coli 0157: H7 aptamer/BiOX/NBC/ITO electrode in an E. coli 0157: H7 dispersion, and conducting incubation, where a detection range is 0.5 to 5×10⁶ CFU/mL.

In the step (B1), a concentration of the BiOX/NBC nanocomposite in the dispersion is 5 mg/mL.

In the step (B3), the E. coli 0157: H7 aptamer has a sequence of ATCCGTCACACCTGCTCTACTGGCCGGCTCAGCATGACTAAGA-AGGAAGTTATGTGG TGTTGGCTCCCGTAT-3′ (SEQ ID NO: 2); and the BSA has a concentration of 1 mmol/L.

In the step (B4), the E. coli 0157: H7 dispersion has a concentration of 0.5 to 5×10⁶ CFU/mL; the xenon light source has an intensity of 25% to 100%; and the incubation is conducted for 0.5 h.

The present disclosure has the following beneficial effects.
1. In the present disclosure, a discarded crayfish shell, crab shell, or tofu residue is used as a raw material to prepare the BiOX/N-doped biochar nanocomposite, which realizes the transformation of a renewable biological resource from waste into treasure.
2. The present disclosure uses a protein existing in the crayfish shell, crab shell, or tofu residue itself to achieve N doping without the addition of an additional nitrogen source.
3. The present disclosure provides a method for preparing a BiOX/N-doped biochar nanocomposite by a microwave method at a low temperature, which involves a simple process, raw materials that are cheap and readily available on the market, and a short cycle and is suitable for industrial production.
4. The present disclosure proposes for the first time that N-doped biochar can effectively improve the absorption and electron transport capacities of BiOI or BiOBr under visible light, and improve the photoelectrochemical performance of BiOI or BiOBr.
5. The present disclosure uses a biochar material as a sensitized carbon material in the field of photoelectrochemistry for the first time.
6. In the present disclosure, the prepared BiOX/N-doped biochar nanocomposite is used as a photoelectric active material to construct a photoelectrochemical sensor, and the photoelectrochemical sensor can be used in the fields of plant nutrient detection and food safety.
7. The photoelectric sensor constructed based on the BiOX/N-doped biochar nanocomposite proposed in the present disclosure achieves the detection of ATP or E. coli.
8. The present disclosure proposes for the first time a sensor for detecting ATP based on a photoelectrochemical signal “on-off-on”.

### BRIEF DESCRIPTION OF THE DRAWINGS

FIG. 1 is an X-ray diffractometry (XRD) pattern of the BiOI/N-doped biochar nanocomposite prepared in Example 3.

FIG. 2 is an infrared (IR) spectrum of the BiOI/N-doped biochar nanocomposite prepared in Example 3.

FIG. 3 is an X-ray photoelectron spectroscopy (XPS) spectrum of the BiOI/N-doped biochar nanocomposite prepared in Example 3.

FIG. 4 shows photocurrent curves of the BiOI/N-doped biochar nanocomposite prepared in Example 3 under different conditions, where curve a shows a photocurrent of a BiOI/NBC/ITO electrode, curve b shows a photocurrent of an aptamer/BiOI/NBC/ITO electrode, and curve c shows a photocurrent of an ATP/aptamer/BiOI/NBC/ITO electrode.

FIG. 5 shows XRD patterns of the BiOBr/NBC nanocomposite prepared in Example 5, where curve a is for a BiOBr nanosheet and curve b is for a BiOBr/NBC nanocomposite.

FIG. 6 is an XPS spectrum of the BiOBr/NBC nanocomposite prepared in Example 5.

FIG. 7 shows photocurrent results of the BiOBr/NBC nanocomposite prepared in Example 5 under different conditions, where A shows a photocurrent intensity generated by an E. coli O157: H7 aptamer/BiOBr/NBC/ITO electrode with the increase in E. coli concentration and B shows the optimal linear range of the E. coli 0157: H7 aptamer/BiOBr/NBC/ITO electrode.

### DETAILED DESCRIPTION OF THE EMBODIMENTS

The technical content and embodiments of the present disclosure are further specifically described in conjunction with the embodiments and accompanying drawings.

### Example 1

A preparation method of a BiOI/N-doped biochar nanocomposite was provided, including the following steps.

### Step 1: Preparation of an N-Doped Biochar

A cleaned crayfish shell (derived from crayfish on the fish market) was placed in an aluminum oxide crucible, a sufficient amount of NaOH was added, and calcination was conducted in a tube furnace with an Ar atmosphere as follows: raising a temperature at 5° C./min from room temperature to 700° C., and holding the temperature for 2 h; and a resulting system was cooled and then washed with distilled water until neutral, and a resulting solid was collected and dried at 80° ° C. for 24 h to obtain the N-doped biochar.

### Step 2: Preparation of an Acidified N-Doped Biochar

The N-doped biochar obtained in the step 1 was dispersed in a mixed solution of HCl and HNO₃ (a volume ratio of HCl to HNO; was 3:1) to obtain a mixed solution A; the mixed solution A was subjected to an ultrasonic treatment for 6 h in an ultrasonic cleaner and then filtered; and a filter residue was washed with a large amount of C₂H₅OH and deionized water, and then dried in an oven at 80° ° C. to obtain the acidified N-doped biochar, which was denoted as NBC.

Step 3: Preparation of the BiOI/N-doped biochar nanocomposite 1 mg of the acidified N-doped biochar obtained in the step 2 and 0.01 mol of Bi(NO₃)_{3·}5H₂O were added to 40 mL of acetic acid, and a resulting mixture was subjected to an ultrasonic treatment for 10 min to obtain a suspension A; under vigorous stirring, a KI aqueous solution (0.01 mol of KI+20 mL of H₂O) was added dropwise to the suspension A (a precipitate was produced) to obtain a mixed solution, the mixed solution was continuously stirred for 30 min, and 25 mL of the mixed solution was taken and transferred to a CEM microwave reactor; a microwave power (MP) was set to 200 W, a reaction temperature (T) was set to 150° C., and a reaction was conducted for 1 h (t); after the reaction was completed, a resulting solid was collected through centrifugation, washed, dispersed in absolute ethanol, dried, and subjected to calcination at 300° C. for 2 h in a tube furnace with a N₂ atmosphere to obtain a BiOI/N-doped biochar nanocomposite, which was denoted as a BiOI/NBC nanocomposite. According to the above process, a monomer BiOI was prepared without the addition of N-doped biochar.

A use of the BiOI/N-doped biochar nanocomposite in the preparation of a photoelectrochemical sensor for detecting ATP was provided, including the following steps.
(1) The BiOI/N-doped biochar nanocomposite was dispersed in DMF to obtain a 5 mg/ml suspension.
(2) 10 μL to 50 μL of the suspension obtained in the step (1) was added to an ITO electrode for modification, the ITO electrode was dried at room temperature to obtain a modified electrode, which was denoted as BiOI/NBC/ITO; and 10 μL to 50 μL of an ATP aptamer solution (an aptamer had a sequence of 5′-ACCTGGGGGAGTATTGCGGAGGAAGGT-3′ (SEQ ID NO: 1)) was drip-coated on the modified electrode to obtain an aptamer/BiOI/NBC/ITO electrode.
(3) 10 μL to 50 μL of an ATP solution with a concentration of 1×10⁻¹² mol/L to 1×10⁻⁵ mol/L was drip-coated on the aptamer/BiOI/NBC/ITO electrode to obtain an ATP/aptamer/BiOI/NBC/ITO electrode; an electrochemical workstation three-electrode system was assembled with the ATP/aptamer/BiOI/NBC/ITO electrode as a working electrode, an SCE as a reference electrode, and a platinum wire as a counter electrode, and under the irradiation of a xenon light source (with an intensity of 25%), photoelectrochemical analysis was conducted; and the photoelectrochemical sensor constructed based on the BiOI/N-doped biochar nanocomposite was used to detect ATP.

### Example 2

A preparation method of a BiOI/N-doped biochar nanocomposite was provided, including the following steps.

### Step 1: Preparation of an N-Doped Biochar

A cleaned crab shell (derived from a crab on the fish market) was placed in an aluminum oxide crucible, a sufficient amount of KOH was added, and calcination was conducted in a tube furnace with an Ar atmosphere as follows: raising a temperature at 5° C./min from room temperature to 700° C., and holding the temperature for 2 h; and a resulting system was cooled and then washed with distilled water until neutral, and a resulting solid was collected and dried at 80ºC for 24 h to obtain the N-doped biochar.

### Step 2: Preparation of an Acidified N-Doped Biochar

The N-doped biochar obtained in the step 1 was dispersed in a mixed solution of HCl and HNO₃ (a volume ratio of HCl to HNO; was 3:1) to obtain a mixed solution A; the mixed solution A was subjected to an ultrasonic treatment for 6 h in an ultrasonic cleaner and then filtered; and a filter residue was washed with a large amount of C₂H₅OH and deionized water, and then dried in an oven at 80° ° C. to obtain the acidified N-doped biochar, which was denoted as NBC.

### Step 3: Preparation of the BiOI/N-Doped Biochar Nanocomposite

20 mg of the acidified N-doped biochar obtained in the step 2 and 0.05 mol of Bi(NO₃)_{3·}5H₂O were added to 40 mL of acetic acid, and a resulting mixture was subjected to an ultrasonic treatment for 10 min to obtain a suspension A; under vigorous stirring, a KI aqueous solution (0.01 mol of KI+20 mL of H₂O) was added dropwise to the suspension A (a precipitate was produced) to obtain a mixed solution, the mixed solution was continuously stirred for 30 min, and 25 mL of the mixed solution was taken and transferred to a CEM microwave reactor; a microwave power (MP) was set to 200 W, a reaction temperature (T) was set to 160° ° C., and a reaction was conducted for 1 h (t); after the reaction was completed, a resulting solid was collected through centrifugation, washed, dispersed in absolute ethanol, dried, and subjected to calcination at 300° C. for 2 h in a tube furnace with a N₂ atmosphere to obtain a BiOI/N-doped biochar nanocomposite, which was denoted as a BiOI/NBC nanocomposite. According to the above process, a monomer BiOI was prepared without the addition of N-doped biochar.

A use of the BiOI/N-doped biochar nanocomposite in the preparation of a photoelectrochemical sensor for detecting ATP was provided, including the following steps.
(1) The BiOI/N-doped biochar nanocomposite was dispersed in DMF to obtain a 5 mg/ml suspension.
(2) 10 μL of the suspension obtained in the step (1) was added to an ITO electrode for modification, the ITO electrode was dried at room temperature to obtain a modified electrode, which was denoted as BiOI/NBC/ITO; and 10 μL of an ATP aptamer solution (an aptamer had a sequence of 5′-ACCTGGGGGAGTATTGCGGAGGAAGGT-3′ (SEQ ID NO: 1)) was drip-coated on the modified electrode to obtain an aptamer/BiOI/NBC/ITO electrode.
(3) 10 μL of an ATP solution with a concentration of 1×10⁻¹² mol/L to 1×10⁻⁵ mol/L was drip-coated on the aptamer/BiOI/NBC/ITO electrode to obtain an ATP/aptamer/BiOI/NBC/ITO electrode; an electrochemical workstation three-electrode system was assembled with the ATP/aptamer/BiOI/NBC/ITO electrode as a working electrode, an SCE as a reference electrode, and a platinum wire as a counter electrode, and under the irradiation of a xenon light source (with an intensity of 75%), photoelectrochemical analysis was conducted; and the photoelectrochemical sensor constructed based on the BiOI/N-doped biochar nanocomposite was used to detect ATP.

### Example 3

A preparation method of a BiOI/N-doped biochar nanocomposite was provided, including the following steps.

### Step 1: Preparation of an N-Doped Biochar

A cleaned crayfish shell (derived from crayfish on the fish market) was placed in an aluminum oxide crucible, a sufficient amount of NaOH was added, and calcination was conducted in a tube furnace with an Ar atmosphere as follows: raising a temperature at 5° C./min from room temperature to 700° C., and holding the temperature for 2 h; and a resulting system was cooled and then washed with distilled water until neutral, and a resulting solid was collected and dried at 80° ° C. for 24 h to obtain the N-doped biochar.

### Step 2: Preparation of an Acidified N-Doped Biochar

The N-doped biochar obtained in the step 1 was dispersed in a mixed solution of HCl and HNO₃ (a volume ratio of HCl to HNO; was 3:1) to obtain a mixed solution A; the mixed solution A was subjected to an ultrasonic treatment for 6 h in an ultrasonic cleaner and then filtered; and a filter residue was washed with a large amount of C₂H₅OH and deionized water, and then dried in an oven at 80° ° C. to obtain the acidified N-doped biochar, which was denoted as NBC.

### Step 3: Preparation of the BiOI/N-Doped Biochar Nanocomposite

10 mg of the acidified N-doped biochar obtained in the step 2 and 0.02 mol of Bi(NO₃)_{3·}5H₂O were added to 40 mL of acetic acid, and a resulting mixture was subjected to an ultrasonic treatment for 10 min to obtain a suspension A; under vigorous stirring, a KI aqueous solution (0.01 mol of KI+20 mL of H₂O) was added dropwise to the suspension A (a precipitate was produced) to obtain a mixed solution, the mixed solution was continuously stirred for 30 min, and 25 mL of the mixed solution was taken and transferred to a CEM microwave reactor; a microwave power (MP) was set to 200 W, a reaction temperature (T) was set to 180° C., and a reaction was conducted for 1 h (t); after the reaction was completed, a resulting solid was collected through centrifugation, washed, dispersed in absolute ethanol, dried, and subjected to calcination at 300° C. for 2 h in a tube furnace with a N₂ atmosphere to obtain a BiOI/N-doped biochar nanocomposite, which was denoted as a BiOI/NBC nanocomposite. According to the above process, a monomer BiOI was prepared without the addition of N-doped biochar.

A use of the BiOI/N-doped biochar nanocomposite in the preparation of a photoelectrochemical sensor for detecting ATP was provided, including the following steps.
(1) The BiOI/N-doped biochar nanocomposite was dispersed in DMF to obtain a 5 mg/ml suspension.
(2) 50 μL of the suspension obtained in the step (1) was added to an ITO electrode for modification, the ITO electrode was dried at room temperature to obtain a modified electrode, which was denoted as BiOI/NBC/ITO; and 50 μL of an ATP aptamer solution (an aptamer had a sequence of 5′-ACCTGGGGGAGTATTGCGGAGGAAGGT-3′ (SEQ ID NO: 1)) was drip-coated on the modified electrode to obtain an aptamer/BiOI/NBC/ITO electrode.
(3) 50 μL of an ATP solution with a concentration of 1×10⁻¹² mol/L to 1×10⁻⁵ mol/L was drip-coated on the aptamer/BiOI/NBC/ITO electrode to obtain an ATP/aptamer/BiOI/NBC/ITO electrode; an electrochemical workstation three-electrode system was assembled with the ATP/aptamer/BiOI/NBC/ITO electrode, an aptamer/BiOI/NBC/ITO electrode, and a BiOI/NBC/ITO electrode respectively as a working electrode, an SCE as a reference electrode, and a platinum wire as a counter electrode, and under the irradiation of a xenon light source (with an intensity of 100%), photoelectrochemical analysis was conducted; and the photoelectrochemical sensor constructed based on the BiOI/N-doped biochar nanocomposite was used to detect ATP.

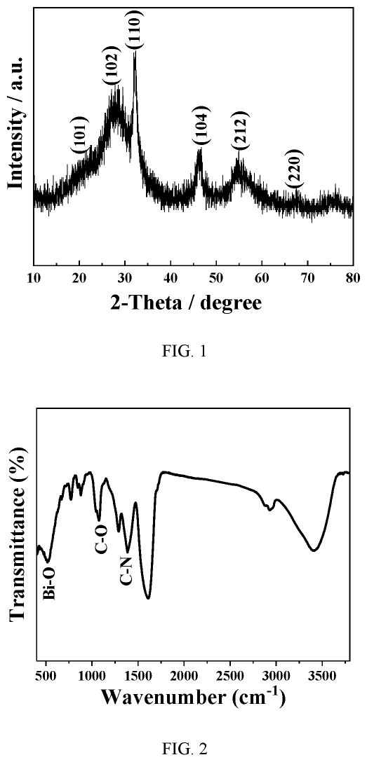

FIG. 1 is an XRD pattern of the BiOI/N-doped biochar nanocomposite. As shown in the figure, the appeared characteristic peaks can correspond to a BiOl standard card (JCPDS NO. 10-0445) of a tetragonal crystal system, and these diffraction peaks belong to crystal planes (101), (102), (110), (104), (212), and (220), respectively. However, compared with the monomer BiOI, NBC-associated characteristic peaks are not observed, which is attributed to a low doping amount of NBC. In addition, there is no impurity peak in the XRD pattern, indicating that the synthesized material has a high crystal quality.

FIG. 2 is an IR spectrum of the BiOI/N-doped biochar nanocomposite prepared in Example 3. As shown in the figure, absorption peaks of the BiOI (curve a) and the BiOI/N-doped biochar nanocomposite (curve b) at 512 cm⁻¹ are attributed to a stretching vibration of Bi—O. In addition, curves a and b have obvious absorption peaks at 1,621 cm⁻¹ and 3,430 cm⁻¹ that are attributed to a stretching vibration of 8(O—H) and a stretching vibration of v(O—H), respectively, and this is due to the absorption of a small amount of water on a surface of the material. Curves b and c show a stretching vibration of C—N and a stretching vibration of C—O at 1,400 cm⁻¹ and 1,078 cm⁻¹, respectively, which can be attributed to the doping of NBCs in BiOI. The above results show that BiOI and NBC are successfully compounded.

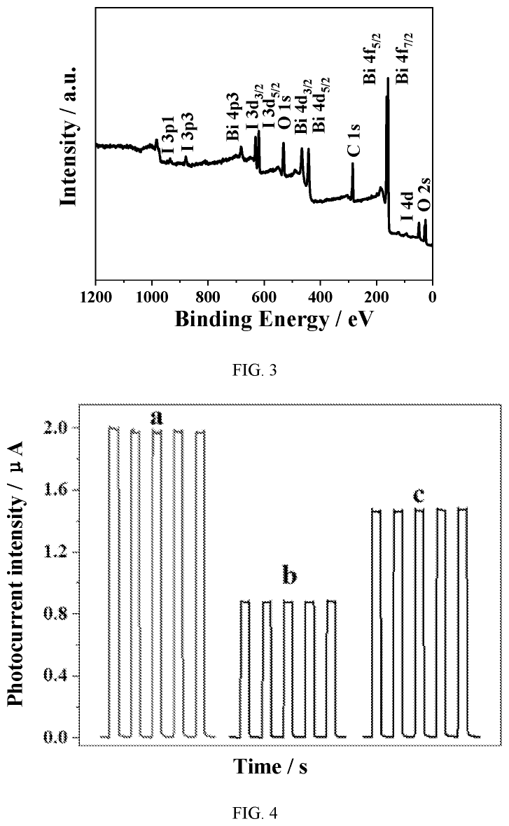

FIG. 3 is an XPS spectrum of the BiOI/N-doped biochar nanocomposite prepared in Example 3. It can be seen from the full spectrum of XPS that the BiOI/N-doped biochar nanocomposite includes Bi, I, C, and O, and similarly, N in NBC is not observed in the full spectrum of XPS, which is due to the fact that a content of N is relatively low compared with other elements and thus N is not easily observed.

FIG. 4 shows a change of a photocurrent signal during a preparation process of a sensor. The BiOI/N-doped biochar nanocomposite-modified electrode (curve a) has a strong photocurrent response due to its efficient charge separation, but the aptamer/BiOI/NBC/ITO modified electrode combining the aptamer (curve b) has a significantly reduced photocurrent, which is due to steric hindrance of the aptamer to hinder the diffusion of electrons towards a surface of the electrode. After the ATP solution is drip-coated on the prepared aptamer/BiOI/NBC/ITO electrode (curve c), the photocurrent is enhanced, and this is mainly because the aptamer on the electrode can specifically recognize ATP and make ATP released from the surface of the material, such that the electron transport hindered by the aptamer can be restored and thus the photocurrent of the sensor can be restored. In this way, the present disclosure realizes the construction of a sensor for detecting ATP based on a photoelectrochemical signal “on-off-on”.

### Example 4

A preparation method of a BiOI/N-doped biochar nanocomposite was provided, including the following steps.

### Step 1: Preparation of an N-Doped Biochar

A cleaned crab shell (derived from a crab on the fish market) was placed in an aluminum oxide crucible, a sufficient amount of KOH was added, and calcination was conducted in a tube furnace with an Ar atmosphere as follows: raising a temperature at 5° C./min from room temperature to 700° C., and holding the temperature for 2 h; and a resulting system was cooled and then washed with distilled water until neutral, and a resulting solid was collected and dried at 80° C. for 24 h to obtain the N-doped biochar.

### Step 2: Preparation of an Acidified N-Doped Biochar

The N-doped biochar obtained in the step 1 was dispersed in a mixed solution of HCl and HNO₃ (a volume ratio of HCl to HNO₃ was 3:1) to obtain a mixed solution A; the mixed solution A was subjected to an ultrasonic treatment for 6 h in an ultrasonic cleaner and then filtered; and a filter residue was washed with a large amount of C₂H₅OH and deionized water, and then dried in an oven at 80° ° C. to obtain the acidified N-doped biochar, which was denoted as NBC.

### Step 3: Preparation of the BiOI/N-Doped Biochar Nanocomposite

5 mg of the acidified N-doped biochar obtained in the step 2 and 0.03 mol of Bi(NO₃)_{3·}5H₂O were added to 40 mL of acetic acid, and a resulting mixture was subjected to an ultrasonic treatment for 10 min to obtain a suspension A; under vigorous stirring, a KI aqueous solution (0.01 mol of KI+20 mL of H₂O) was added dropwise to the suspension A (a precipitate was produced) to obtain a mixed solution, the mixed solution was continuously stirred for 30 min, and 25 mL of the mixed solution was taken and transferred to a CEM microwave reactor; a microwave power (MP) was set to 200 W, a reaction temperature (T) was set to 170° C., and a reaction was conducted for 1 h (t); after the reaction was completed, a resulting solid was collected through centrifugation, washed, dispersed in absolute ethanol, dried, and subjected to calcination at 300° C. for 2 h in a tube furnace with a N₂ atmosphere to obtain a BiOI/N-doped biochar nanocomposite, which was denoted as a BiOI/NBC nanocomposite. According to the above process, a monomer BiOI was prepared without the addition of N-doped biochar.

A use of the BiOI/N-doped biochar nanocomposite in the preparation of a photoelectrochemical sensor for detecting ATP was provided, including the following steps.
(1) The BiOI/N-doped biochar nanocomposite was dispersed in DMF to obtain a 5 mg/ml suspension.
(2) 30 μL of the suspension obtained in the step (1) was added to an ITO electrode for modification, the ITO electrode was dried at room temperature to obtain a modified electrode, which was denoted as BiOI/NBC/ITO; and 30 μL of an ATP aptamer solution (an aptamer had a sequence of 5′-ACCTGGGGGAGTATTGCGGAGGAAGGT-3′ (SEQ ID NO: 1)) was drip-coated on the modified electrode to obtain an aptamer/BiOI/NBC/ITO electrode.
(3) 30 μL of an ATP solution with a concentration of 1×10⁻¹² mol/L to 1×10⁻⁵ mol/L was drip-coated on the aptamer/BiOI/NBC/ITO electrode to obtain an ATP/aptamer/BiOI/NBC/ITO electrode; an electrochemical workstation three-electrode system was assembled with the ATP/aptamer/BiOI/NBC/ITO electrode as a working electrode, an SCE as a reference electrode, and a platinum wire as a counter electrode, and under the irradiation of a xenon light source (with an intensity of 50%), photoelectrochemical analysis was conducted; and the photoelectrochemical sensor constructed based on the BiOI/N-doped biochar nanocomposite was used to detect ATP.

### Example 5

A preparation method of a BiOBr/N-doped biochar nanocomposite was provided, including the following steps.

### Step 1: Preparation of an N-Doped Biochar

A tofu residue (purchased from the bean product market) was placed in an aluminum oxide crucible, then NaOH (keeping NaOH sufficient) was added, and calcination was conducted in a tube furnace with an Ar atmosphere (raising a temperature at 5° C. min-1 from room temperature to 700° C., and holding the temperature for 2 h); and a resulting system was cooled and then washed with distilled water until neutral, and a resulting solid was collected and dried at 80° C. for 24 h to obtain the N-doped biochar.

### Step 2: Preparation of an Acidified N-Doped Biochar

The N-doped biochar obtained in the step 1 was added to a mixed solution of HCl and HNO₃ (a volume ratio of HCl to HNO₃ was 3:1) to obtain a mixed solution A; the mixed solution A was subjected to an ultrasonic treatment for 6 h in an ultrasonic cleaner and then filtered; and a filter residue was washed with a large amount of C₂H₅OH and deionized water, and then dried in an oven at 80° ° C. to obtain the acidified N-doped biochar, which was denoted as NBC.

### Step 3: Preparation of the BiOBr/N-Doped Biochar Nanocomposite

10 mg of the acidified N-doped biochar obtained in the step 2 and 0.03 mol of Bi(NO₃)₃·5H₂O were added to 40 mL of acetic acid, and a resulting mixture was subjected to an ultrasonic treatment for 10 min to obtain a suspension A; under vigorous stirring, a KBr aqueous solution (0.01 mol of KBr+20 mL of H₂O) was added dropwise to the suspension A (a precipitate was produced) to obtain a mixed solution, the mixed solution was continuously stirred for 30 min, and 25 mL of the mixed solution was taken and transferred to a CEM microwave reactor; a microwave power (MP) was set to 200 W, a reaction temperature (T) was set to 180° C., and a reaction was conducted for 1 h (t); after the reaction was completed, a resulting solid was collected through centrifugation, washed, dispersed in absolute ethanol, dried, and subjected to calcination at 300° C. for 2 h in a tube furnace with a N₂ atmosphere to obtain a BiOBr/NBC nanocomposite. According to the above process, a monomer BiOBr was prepared without the addition of N-doped biochar, and a BiOBr nanosheet was actually obtained.

A use of the BiOBr/NBC nanocomposite in the preparation of a photoelectrochemical sensor for detecting E. coli was provided, including the following steps.

### (1) Preparation of a BiOBr/NBC Nanocomposite Dispersion

The prepared BiOBr/NBC nanocomposite was dispersed in DMF to obtain the dispersion with a concentration of 5 mg/mL.

### (2) Surface Pretreatment of an ITO Electrode

A 1×0.5 cm² ITO electrode was first boiled in a 1 mol/L sodium hydroxide solution for 15 min to 20 min, then subjected to ultrasonic cleaning with acetone, double-distilled water, and ethanol successively, and blow-dried with nitrogen for later use.

### (3) Construction of a Photoelectrochemical Biological Interface

20 μL of the BiOBr/NBC nanocomposite dispersion prepared in the step (1) was taken with a microsyringe and drip-coated on a surface of the ITO electrode obtained in the step (2), and the ITO electrode was oven-dried with an IR lamp to obtain an electrode denoted as BiOBr/NBC/ITO; 8 μL of GA was drip-coated on a surface of the BiOBr/NBC/ITO, and 8 μL of a 4 μmol/L E. coli 0157: H7 (E. coli 0157: H7) aptamer solution was added to the surface of the electrode for modification to obtain an E. coli 0157: H7 aptamer/BiOBr/NBC/ITO electrode;

the electrode was stored overnight in a 4ºC refrigerator, then rinsed with PBS (pH=7.0, concentration: 0.1 mol/L) multiple times to remove the physical adsorption, and dried in a N₂ atmosphere; and 8 μL of BSA (1 mmol/L) was drip-coated on a surface of the electrode, and the electrode was allowed to stand at room temperature for 1 h to block non-specific adsorption sites on the modified electrode and finally rinsed with UPW to remove the unbound aptamer. An E. coli 0157 H7 had a aptamer sequence of

(SEQ ID NO: 2)
ATCCGTCACACCTGCTCTACTGGCCGGCTCAGCATGACTAAGA-
AGGAAGTTATGTGGTGTTGGCTCCCGTAT-3′.

(4) Correspondence Between an E. coli 0157: H7 Concentration and a PEC Signal

An electrochemical workstation three-electrode system was assembled with the E. coli O157: H7 aptamer/BiOBr/NBC/ITO electrode obtained in the step (3) placed in 5 mL of PBS (pH=7 to 8, concentration: 0.1 mol/L) as a working electrode at a bias voltage of 0.0 V, a platinum wire electrode as a counter electrode, and an SCE as a reference electrode, and under the irradiation of a xenon light source (with a light intensity of 75%), a photoelectrochemical signal (PEC) was acquired by an i-t curve method; and the E. coli 0157: H7 aptamer/BiOBr/NBC/ITO electrode was immersed in an E. coli 0157: H7 dispersion at different concentrations and then incubated for 0.5 h, and then detection was conducted.

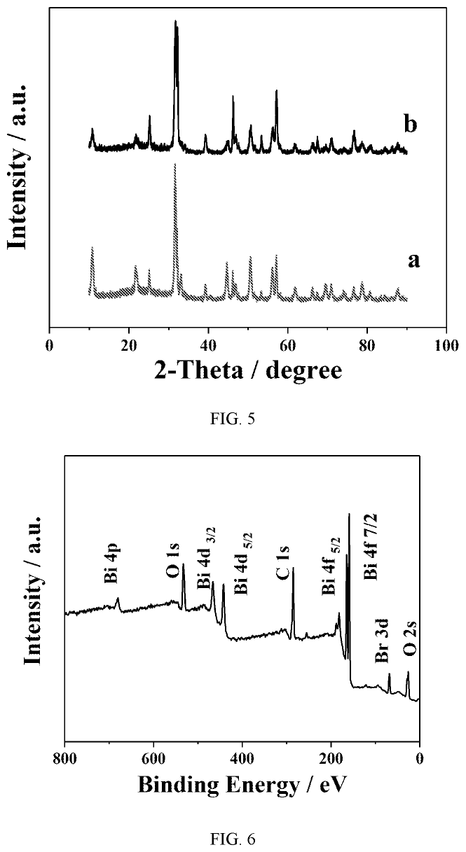

FIG. 5 shows XRD patterns of the BiOBr nanosheet (curve a) and the BiOBr/NBC nanocomposite (curve b). As shown in the figure, the appeared characteristic peaks of all materials can correspond to a BiOBr standard card (JCPDS No. 73-2061) of a tetragonal crystal system, and these diffraction peaks belong to crystal planes (011), (012), (110), (112), (020), (014), (211), (212), (220), (124), and (032), respectively. In addition, no impurity peak appears in the XRD patterns, indicating that a monocrystalline BiOBr nanosheet of a tetragonal crystal system is obtained by the solvothermal method, and the introduction of biochar does not affect a crystal structure of BiOBr. However, biochar-associated characteristic peaks are not observed, which is attributed to a low doping amount of biochar.

Through XPS characterization, the chemical composition and electronic structure of the BiOBr/NBC nanocomposite were further investigated. FIG. 6 shows a full XPS spectrum of the BiOBr/NBC nanocomposite, and it can be seen from the figure that the BiOBr/NBC nanocomposite includes Bi, Br, O, and C.

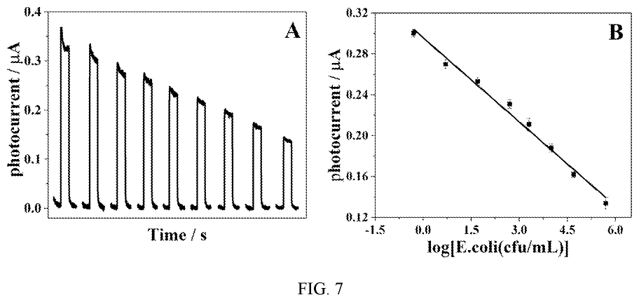

E. coli 0157: H7 concentrations to be tested are 0 CFU/mL, 0.5 CFU/mL, 5 CFU/mL, 50 CFU/mL, 500 CFU/mL, 1,000 CFU/mL, 2,000 CFU/mL, 5×10⁵ CFU/mL, and 5×10⁶ CFU/mL. As shown in A of FIG. 7, a photocurrent intensity decreases with the increase in E. coli concentration. As shown in B of FIG. 7, a standard curve is plotted with the photocurrent intensity (I) and different E. coli concentration change values, and it can be known that an optimal linear range is 0.5 CFU/mL to 5×10⁶ CFU/mL and a minimum detection limit is 0.17 CFU/mL. In conclusion, the photoelectrochemical aptamer sensor of the present disclosure can be used for sensitive detection of E. coli.

What is claimed is:

1. A preparation method of a BiOX/N-doped biochar nanocomposite, comprising the following steps:
step 1: preparation of an N-doped biochar by placing a cleaned crayfish shell, crab shell, or tofu residue in an aluminum oxide crucible, adding a sufficient amount of a strong alkali, and conducting calcination in a tube furnace with an inert atmosphere; and cooling a resulting system, washing the system until neutral, and collecting and drying a resulting solid to obtain the N-doped biochar;
step 2: preparation of an acidified N-doped biochar by dispersing the N-doped biochar obtained in the step 1 in a mixed solution of HCl and HNO₃ to obtain a mixed solution A; subjecting the mixed solution A to an ultrasonic treatment in an ultrasonic cleaner, and filtering; and washing a filter residue, and drying the filter residue in an oven to obtain the acidified N-doped biochar, which is denoted as NBC; and
step 3: preparation of the BiOX/N-doped biochar nanocomposite by adding the acidified N-doped biochar obtained in the step 2 and Bi(NO₃)₃·5H₂O to acetic acid, and subjecting a resulting mixture to an ultrasonic treatment to obtain a suspension A; under vigorous stirring, adding a KX aqueous solution dropwise to the suspension A to obtain a mixed solution; continuously stirring the mixed solution, transferring the mixed solution to a CEM microwave reactor, setting a microwave power, and conducting a reaction at a constant temperature; after the reaction is completed, collecting a resulting solid through centrifugation, and washing the solid; and dispersing the solid in absolute ethanol, drying, and subjecting a dried product to calcination in a tube furnace with a N₂ atmosphere to obtain a BiOX/N-doped biochar composite, which is denoted as a BiOX/NBC nanocomposite, wherein X is I or Br.

2. The preparation method according to claim 1, wherein in the step 1, the strong alkali is NaOH or KOH; the inert atmosphere is Ar; the calcination is conducted as follows: raising a temperature at 5° C./min from room temperature to 700° C., and holding the temperature for 2 h; and the drying is conducted at 80° C. for 24 h.

3. The preparation method according to claim 1, wherein in the step 2, in the mixed solution of HCl and HNO₃, a volume ratio of the HCl to the HNO₃ is 3:1; and the ultrasonic treatment is conducted for 6 h.

4. The preparation method according to claim 1, wherein in the step 3, in the suspension A, the acidified N-doped biochar, the Bi(NO₃)₃·5H₂O, and the acetic acid are used in a ratio of (1-20) mg:(0.01-0.05) mol: 40 mL; and the continuous stirring is conducted for 30 min.

5. The preparation method according to claim 1, wherein in the step 3, a concentration of KX in the KX aqueous solution is 0.5 mol/L; and a volume ratio of the suspension A to the KX aqueous solution is 2:1.

6. The preparation method according to claim 1, wherein in the step 3, the reaction is conducted at the constant temperature of 150° ° C. to 180° C. and the microwave power of 200 W for 1 h; and the calcination in the tube furnace is conducted at 300° ° C. for 2 h.

7. A use of a BiOX/N-doped biochar nanocomposite prepared according to claim 1 in preparation of a photoelectrochemical sensor for detecting adenosine triphosphate (ATP).

8. A use of a BiOX/N-doped biochar nanocomposite prepared according to claim 1 in preparation of a photoelectrochemical sensor for detecting Escherichia coli.

9. A use of a BiOX/N-doped biochar nanocomposite prepared according to claim 2 in preparation of a photoelectrochemical sensor for detecting adenosine triphosphate (ATP).

10. A use of a BiOX/N-doped biochar nanocomposite prepared according to claim 3 in preparation of a photoelectrochemical sensor for detecting adenosine triphosphate (ATP).

11. A use of a BiOX/N-doped biochar nanocomposite prepared according to claim 4 in preparation of a photoelectrochemical sensor for detecting adenosine triphosphate (ATP).

12. A use of a BiOX/N-doped biochar nanocomposite prepared according to claim 5 in preparation of a photoelectrochemical sensor for detecting adenosine triphosphate (ATP).

13. A use of a BiOX/N-doped biochar nanocomposite prepared according to claim 6 in preparation of a photoelectrochemical sensor for detecting adenosine triphosphate (ATP).

14. A use of a BiOX/N-doped biochar nanocomposite prepared according to claim 2 in preparation of a photoelectrochemical sensor for detecting Escherichia coli.

15. A use of a BiOX/N-doped biochar nanocomposite prepared according to claim 3 in preparation of a photoelectrochemical sensor for detecting Escherichia coli.

16. A use of a BiOX/N-doped biochar nanocomposite prepared according to claim 4 in preparation of a photoelectrochemical sensor for detecting Escherichia coli.

17. A use of a BiOX/N-doped biochar nanocomposite prepared according to claim 5 in preparation of a photoelectrochemical sensor for detecting Escherichia coli.

18. A use of a BiOX/N-doped biochar nanocomposite prepared according to claim 6 in preparation of a photoelectrochemical sensor for detecting Escherichia coli.
